(19) United States
(12) Patent Application Publication
(10) Pub. No.: US 2026/0287784 A1
(43) Pub. Date: Sep. 24, 2026

(54) EXPLORATION OF DISSOLVED MINERALS USING PALEOGEOGRAPHIC RECONSTRUCTION

(71) Applicant: SAUDI ARABIAN OIL COMPANY, Dhahran (SA)

(72) Inventors: Saleh ALQAHTANI, Dhahran (SA); Waleed SAEED, Dhahran (SA); Abdullah WAHBI, Dhahran (SA)

(73) Assignee: SAUDI ARABIAN OIL COMPANY, Dhahran (SA)

(21) Appl. No.: 19/087,188

(22) Filed: Mar. 21, 2025

(51) Int. Cl. G01V 99/00; E21B 43/29; E21B 49/00; E21B 49/02; G01N 33/18

(52) U.S. Cl. CPC G01V 99/00; E21B 43/29; G01N 33/18; E21B 49/005; E21B 49/02

(57) ABSTRACT

A computer-implemented method for identifying resource sinks of water-soluble minerals includes obtaining geological data and paleo-climate data for a region of interest, the geological data including surface data and subsurface data, identifying a potential source rock and a corresponding surface of interest from the geological data, the potential source rocks including water-soluble minerals, reconstructing a paleo-topography of the surface of interest for a paleogeological timespan in which the potential source rock is exposed to weathering via the paleo-climate data, generating a paleo-flow drainage network for the surface of interest to identify streams and catchment boundaries for the region of interest during the paleogeological era, and identifying a possible resource sink from the potential source rock, paleo-topography, and paleo-flow drainage network, wherein the possible resource sink is a paleo-low area into which one or more mineral-rich streams flows in the paleo-flow drainage network.

### FIELD OF THE DISCLOSURE

The present disclosure relates generally to the exploration of water-dissolved minerals in subsurface locations.

### BACKGROUND OF THE DISCLOSURE

Open pit mining and tunneling operations are commonly employed for locating and exposing subterranean sources of valuable minerals. Traditional mining methods are used with identified ore veins and source rocks to provide access to large deposits of valuable metals and other resources. However, the locations and surrounding environments of these conventionally mined ore veins and source rocks can lead to unforeseen difficulties or practically-inaccessible minerals. In some cases, the environmental or human costs of extracting minerals from subterranean locations can prohibit access to large ore veins. Further, while ore veins and source rocks are a major focus of mineral exploration and extraction operations, additional mineral sources can be commonly overlooked in traditional mining endeavors.

However, the weathering over time of source rocks and ore veins can provide additional mineral sources that can be viably mined for valuable resources. Long-term, sustained weathering and erosion of source rocks can enable the leaching or dissolution of ores into water sources. As such, over these sustained periods, substantial minerals and resources can be transported via runoff and waterways to coalesce into a mineral sink at a distance from the source rocks or ore veins. These mineral sinks can be difficult to locate via traditional exploration methods, but can offer novel sources of valuable minerals that are more readily-accessible and currently untapped. However, these mineral sinks are often buried under several layers of new sedimentary units, such that additional effort must be used for the location and extraction of the valuable minerals transported away via weathering and runoff.

Accordingly, systems and methods for accurately locating and estimating contents of subterranean mineral sinks are desirable to enhance mineral recovery from mining operations.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In an embodiment consistent with the present disclosure, a computer-implemented method for identifying resource sinks of water-soluble minerals includes obtaining geological data and paleo-climate data for a region of interest, the geological data including surface data and subsurface data, identifying a potential source rock and a corresponding surface of interest from the geological data, the potential source rocks including water-soluble minerals, reconstructing a paleo-topography of the surface of interest for a paleogeological timespan in which the potential source rock is exposed to weathering via the paleo-climate data, generating a paleo-flow drainage network for the surface of interest to identify streams and catchment boundaries for the region of interest during the paleogeological era, and identifying a possible resource sink from the potential source rock, paleo-topography, and paleo-flow drainage network, wherein the possible resource sink is a paleo-low area into which one or more mineral-rich streams flows in the paleo-flow drainage network.

In another embodiment, a system for locating and sampling resource sinks includes a mineral sink identification engine, implemented on at least one processor, operable to receive geological data and paleo-climate data to identify possible resource sinks. The mineral sink identification engine includes a source rock identification module operable to receive the geological data and identify potential source rocks for valuable minerals and corresponding surfaces of interest, a paleo-topographic reconstruction module operable to reconstruct a paleo-topography of the surfaces of interest wherein the potential source rocks are exposed to open air, a paleo-flow drainage network module operable to construct a paleo-flow drainage network of the surfaces of interest using the geological data and the paleo-climate data, and a paleo-low and sink determination module operable to utilize the potential source rocks, the paleo-topography, and the paleo-flow drainage network to identify the possible resource sinks. The system further includes an identified sink screener operable to extract and test a sample from each possible resource sink for an evaluation of valuable minerals therein.

In a further embodiment, a non-transitory computer-readable medium stores machine-readable instructions, which, when executed by a processor of an electronic device, cause the electronic device to identify a potential source rock and a corresponding surface of interest from available geological data, the potential source rocks including water-soluble minerals, reconstruct a paleo-topography of the surface of interest for a paleogeological timespan in which the potential source rock is exposed to weathering, and generate a paleo-flow drainage network for the surface of interest to identify streams and catchment boundaries for a region of interest during the paleogeological era. The machine-readable instructions further cause the electronic device to identify a possible resource sink from the potential source rock, paleo-topography, and paleo-flow drainage network, wherein the possible resource sink is a paleo-low area into which one or more mineral-rich streams flows in the paleo-flow drainage network, extract and assess, via an identified sink screener, a water sample from the possible resource sink to determine an estimated value of total dissolved solids in the possible resource sink, and initiate a mining operation of the possible resource sink in response to the estimated value of total dissolved solids exceeding a pre-determined threshold.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a schematic view of an example system for locating and sampling resource sinks of water-soluble minerals using paleo-data, according to an embodiment of the present disclosure.

FIG. 2 is a schematic view of an example geological cross-section for identification of potential source rocks buried under one or more sedimentary units, according to an embodiment of the present disclosure.

FIG. 3 is an example weathering plot depicting weathering intensity for a region of interest over millions of years, according to an embodiment of the present disclosure.

FIG. 4 is an example reconstruction of paleo-topography for the region of interest, according to an embodiment of the present disclosure.

FIG. 5 is an example superimposition of the various paleo-data and maps to construct a final paleo-topography with estimated historical flow, according to an embodiment of the present disclosure.

FIG. 6 illustrates a method for identifying, testing, and mining resource sinks of water-soluble minerals developed from the weathering of potential source rocks with water-soluble minerals, according to one or more embodiments of the present disclosure.

FIG. 7 illustrates one example of a computer system that can be employed to execute one or more embodiments of the present disclosure.

### DETAILED DESCRIPTION

Embodiments of the present disclosure will now be described in detail with reference to the accompanying Figures. Like elements in the various Figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure relates generally to the exploration of water-dissolved minerals in subsurface locations, and, more particularly, to leveraging paleogeographic and paleotopographic reconstructions to identify sweetspots of water-dissolved minerals. Embodiments disclosed herein include systems and methods for identifying potential source rocks from seismic and geological data, wherein the potential source rocks can include water-soluble minerals. The disclosed systems and methods can simulate the exposure of these potential source rocks to weather effects during various geological eras, while further predicting the flow of water-soluble minerals from these weathered source rocks. Using the systems and methods of this disclosure, the flow of these minerals can be predicted using reconstructed topographies and drainage networks, such that an estimated representation of the Earth's surface can be constructed for the surface of interest. The combined paleo-data can enable the determination of a possible resource sink in which the dissolved minerals can have pooled and coalesced to form resource deposits for mining operations.

The disclosed systems and methods can further enable the sampling and testing of water samples from identified, possible resource sinks. Thus, the disclosed systems and methods can utilize these water samples to test for salinity of the possible resource sink, which can be leveraged to estimate the total dissolved solids within the possible resource sink. For possible resource sinks with levels of total dissolved solids above a predetermined threshold, the possible resource sink can be identified as a mineral deposit. Thus, the disclosed embodiments can further include the initiation of mining operations within the possible resource sink to begin extraction of these dissolved and coalesced minerals. Through the use of the disclosed systems and methods, previously unexplored mineral deposits can be identified and tested without costly, time-consuming exploration operations and conventional extraction methods.

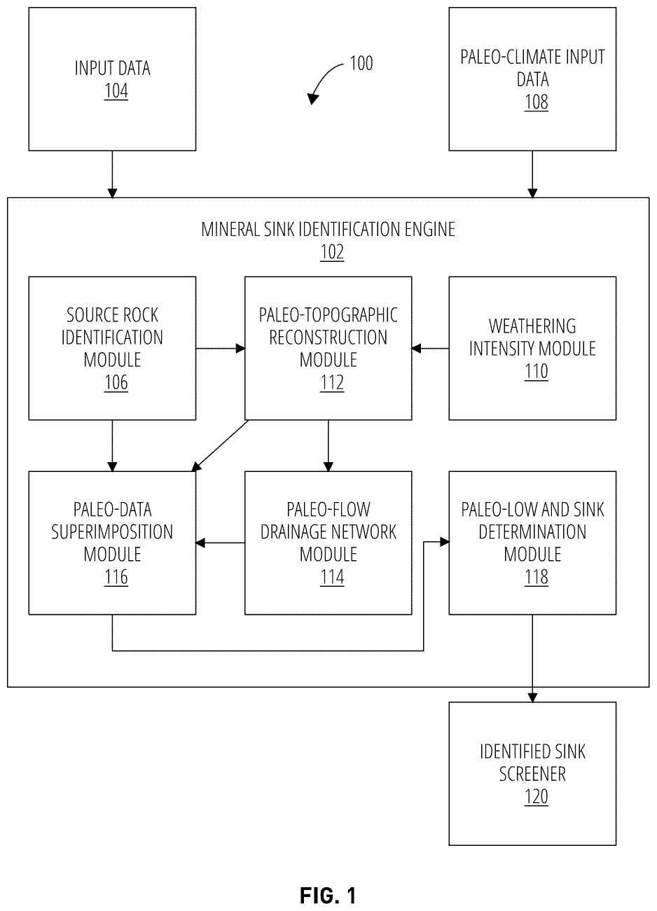

FIG. 1 is a schematic view of an example system 100 for locating and sampling resource sinks of water-soluble minerals using paleo-data, according to an embodiment of the present disclosure. System 100 includes a mineral sink identification engine 102 operable to receive input data 104 for use in a source rock identification module 106, operable to identify potential source rocks in geological data. Mineral sink identification engine 102 can further receive paleo-climate input data 108 for use in a weathering intensity module 110 of mineral sink identification engine 102, such that weathering intensity module 110 can identify periods of intense weathering. Mineral sink identification engine 102 can include a paleo-topographic reconstruction module 112 and a paleo-flow drainage network module 114 for providing topological and flow data for the region of interest to be used in identifying possible resource sinks. Mineral sink identification engine 102 can further include a paleo-data superimposition module 116 operable to superimpose the various paleo-data, and the superimposed data can be received by a paleo-low and sink determination module 118 of mineral sink identification engine 102. Paleo-low and sink determination module 118 can utilize the superimposed paleo-data to identify the possible resource sinks in the region of interest for further testing. As such, system 100 can further include an identified sink screener 120 coupled to mineral sink identification engine 102 for sampling and testing the identified possible resource sinks.

System 100 can enable the identification, location, and sampling of a possible resource sink that includes valuable minerals eroded from the potential source rock over millions of years. System 100 can accordingly leverage various paleo-data and traditional geological data to identify and reconstruct the estimated surface of the Earth during identified timespans using mineral sink identification engine 102. As such, mineral sink identification engine 102 can receive input data 104 for the processes of identification, location, and sampling. Input data 104 can include both surface and subsurface geological data, including seismic data, which can be utilized in identifying potential source rocks therein. In some embodiments, input data 104 can include surface geology regional maps, outcrop maps, imagery maps, seismic attributes, well logs, extracted cores, drill cuttings, or a combination thereof.

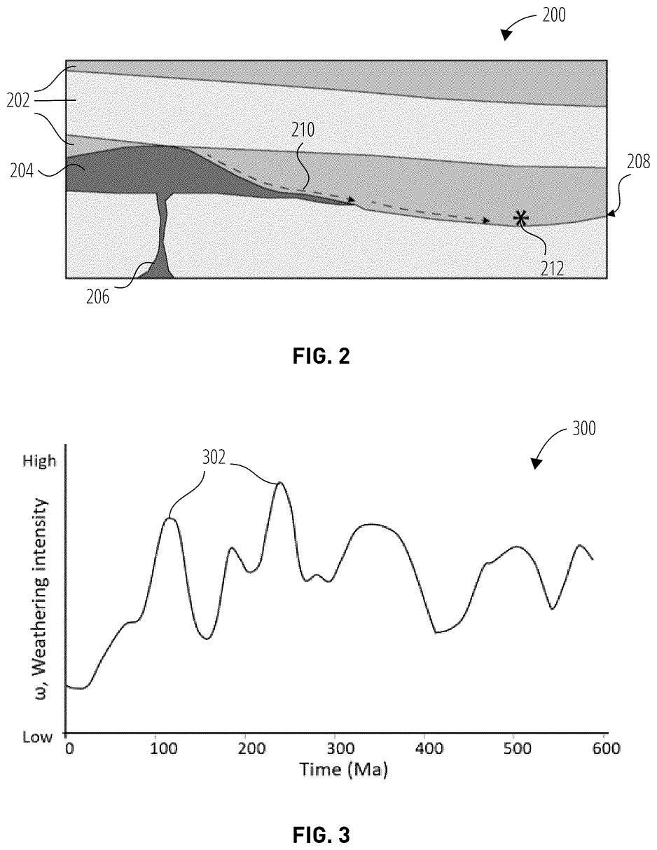

Mineral sink identification engine 102 can include source rock identification module 106, which can utilize received input data 104 to analyze and extract subsurface features for further use in mineral sink identification engine 102. Source rock identification module 106 can utilize geological cross-sections and seismic data of input data 104 for the region of interest, and can identify the sedimentary units and potential source rocks within the geological cross-sections (see FIG. 2). In some embodiments, source rock identification module 106 can further identify a surface of interest corresponding to an identified potential source rock within the geological cross-section, such that the surface of interest can correlate to a specific, or estimated, timespan of surface exposure. Exposure of potential source rocks can occur during deposition (i.e., prior to burial), during volcanic extrusions, or due to exhumation by processes involving erosion, uplift, and/or tectonic activities. The age, and time and duration of exposure for the potential source rock can be estimated and extracted via source rock identification module 106, such that these parameters can be employed in reconstruction efforts of mineral sink identification engine 102. As a non-limiting example, the potential source rocks identified by source rock identification module 106 can include magmatic rocks, which can include felsic and mafic igneous intrusions, as well as volcanic rocks, and a variety of pegmatite minerals such as spodumene, lepidolite, and amblygonite, among others.

In some embodiments, mineral sink identification engine 102 can further obtain or receive paleo-climate input data 108 related to the region of interest for a timespan extending for thousands to millions of years. In these embodiments, paleo-climate input data 108 can include historical humidity values and estimated precipitation for various time steps, such as geological eras or mega-annums. Paleo-climate input data 108 can be utilized in mineral sink identification engine 102, via at least weathering intensity module 110, which can receive paleo-climate input data 108 and determine periods of increased historical humidity values and/or precipitation levels. These increased historical humidity values can result in increased weathering intensities and erosion rates of the potential source rocks, such that increased leaching of valuable minerals into groundwater can occur during these periods. In some embodiments, weathering intensity module 110 can cross-correlate between paleo-climate input data 108 and geological data utilized in source rock identification module 106 to identify overlapping periods wherein there is increased humidity and the potential source rock is exposed. Thus, weathering intensity module 110 can utilize combined input data 104 and paleo-climate input data 108 to identify periods of leaching and erosion of valuable minerals from the potential source rocks into a drainage network (see FIG. 3).

Mineral sink identification engine 102 can further include a paleo-topographic reconstruction module 112 that uses identified eras and potential source rocks for the region of interest, along with input data 104, to reconstruct the topography of the region of interest during the identified era. In some embodiments, paleo-topographic reconstruction module 112 can reconstruct a monotonic subsidence history for the region of interest, via the use of backstripping and decompaction of buried rock units identified by source rock identification module 106. These embodiments can include regions of interest with simple spatio-temporal parameters, such that the reconstruction of the region of interest during the desired timespan can be rapidly performed. In further embodiments, however, paleo-topographic reconstruction module 112 can produce full structural reconstructions of the paleo-topography. In these embodiments, paleo-topographic reconstruction module 112 can account for faults and/or major unconformities of the stratigraphy to produce estimated paleo-topography for the region of interest (see FIG. 4). In further embodiments still, paleo-topographic reconstruction module 112 can directly receive paleo-geography and/or depositional models for the region of interest during the desired timespan to enable rapid and simplistic generation of paleo-topography without extensive reconstruction.

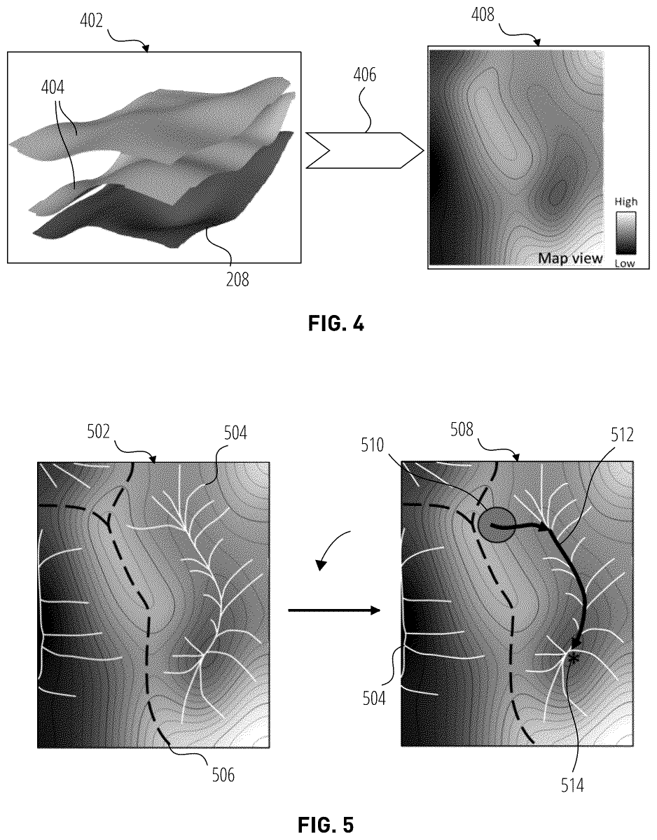

Using the reconstructed paleo-topography, including paleo-highs and lows, mineral sink identification engine 102 can leverage paleo-flow drainage network module 114 to reconstruct a possible drainage network for the paleo-topography. Paleo-flow drainage network module 114 can leverage the reconstructed paleo-topography to determine and identify catchment boundaries of the region of interest, such that any water flow within each catchment boundary can flow to a common body of water. Further, paleo-flow drainage network module 114 can identify any streams within each catchment boundary to determine the full drainage network for the region of interest. The identified streams can accordingly represent any areas of the paleo-topography that can exhibit high surface water accumulation compared to the surrounding area (see FIG. 5).

After the various reconstructions of paleo-data via the previously discussed modules, mineral sink identification engine 102 can access paleo-data superimposition module 116 for overlaying the paleo-data onto a single map. Paleo-data superimposition module 116 can align and superimpose the paleo-topography, potential source rocks, and reconstructed paleo-flow drainage network for the region of interest during the desired timespan. Paleo-data superimposition module 116 can further receive gross-depositional environment data and facies maps to be further overlaid against the paleo-data, such that a robust geological assessment of the region of interest can be provided on a single map.

Mineral sink identification engine 102 can provide the single map produced by paleo-data superimposition module 116 to paleo-low and sink determination module 118, which can accordingly identify patterns and connectivity between potential source rocks and streams of the paleo-flow drainage network. Paleo-low and sink determination module 118 can identify paleo-lows in fluid communication with the potential source rocks on the paleo-topography, with additional focus provided for proper flow directions from paleo-highs to paleo-lows. Paleo-low and sink determination module 118 can identify specific streams and catchments of the paleo-flow drainage network of interest, and these highlighted streams can be compared against the paleo-topography to determine any paleo-lows in which the highlighted streams can converge. Paleo-low and sink determination module 118 can identify the paleo-low most-favored for collecting flow from the highlighted streams, and can extract the location as a possible resource sink for any water-soluble minerals (see FIG. 5). In some embodiments, paleo-low and sink determination module 118 can then back out a present-day subterranean location corresponding to the possible resource sink, such that the possible resource sink can be subsequently located.

In some embodiments, system 100 can include identified sink screener 120 communicably coupled with mineral sink identification engine 102. Identified sink screener 120 can receive the present-day subterranean location for the possible resource sink, and can accordingly extract a fluid sample from the subterranean location. In some embodiments, identified sink screener 120 can include mining or drilling equipment operable to reach the subterranean location and extract the fluid sample. Identified sink screener 120 can screen the fluid sample for a salinity value of the subterranean location, such that the salinity value can advise the level of total dissolved solids in the possible resource sink. Increased salinity can suggest increased levels of total dissolved solids, and higher levels of total dissolved solids increases the likelihood of the possible resource sink including valuable minerals therein. Identified sink screener 120 can further perform additional exploratory and mining operations for verifying the existence and concentration of valuable minerals within the possible resource sink, and can further interface with mining equipment operable to initiate and perform mineral extraction operations on the possible resource sink after verification.

FIG. 2 is a schematic view of an example geological cross-section 200 for identification of potential source rocks buried under one or more sedimentary units 202, according to an embodiment of the present disclosure. Sedimentary units 202 can represent various surfaces and eras of deposition in geological cross-section 200, such that any divisions between sedimentary units 202 can be identified as different eras or geological changes in the region of interest. In geological cross-section 200, a source rock 204 is identifiable as volcanic rock, such as basalt, in line with one or more sedimentary units 202. While source rock 204 can be identified based upon composition or appearance in geological cross-section 200, source rock 204 can be further identified via a connection to an igneous intrusion 206, which penetrates through at least one sedimentary unit 202. Igneous intrusion 206 can represent magmatic flow through geological cross-section 200, which can have solidified into source rock 204 prior to reaching a top surface.

With an identified source rock 204 of the geological cross-section 200, the connected dividing line between sedimentary units 202 can be selected as an identified surface 208. Identified surface 208 can represent the topography or composition of the region of interest in a prior geological era. As such, identified surface 208 can be similar to the surface of the Earth at the time in which source rock 204 was exposed to open air and/or weathering effects. As discussed above, exposure of source rock 204 can occur during deposition, during volcanic extrusions, or due to exhumation by processes involving erosion, uplift, and/or tectonic activities.

Using geological cross-section 200, a crude surface water flow 210 can further be identified, over which any water-dissolved minerals can have traveled away from source rock 204. Surface water flow 210 can advise a possible location of a paleo-low 212 in geological cross-section 200, such that the water-dissolved can coalesce within paleo-low 212.

FIG. 3 is an example weathering plot 300 depicting weathering intensity for a region of interest over millions of years, according to an embodiment of the present disclosure. Weathering plot 300 can include a weighted value of the weathering intensity on the y-axis, such that the weathering intensity can account for historical humidity values, estimated precipitation levels, and other paleo-climate data (e.g., paleo-climate input data 108). Weathering plot 300 can provide this weighted value of the weathering intensity over time, with the x-axis including discretized time for the desired periods of analysis. In the illustrated embodiment, the x-axis can be seen to be in units of mega-annums (Ma), such that hundreds of millions of years are assessed and displayed in weathering plot 300 (e.g., via weathering intensity module 110). Using weathering plot 300, a plurality of weathering peaks 302 can be identified for the region of interest, correlating to timespans of intense weathering that can include significant leaching of water-soluble minerals from potential source rocks.

FIG. 4 is an example reconstruction of paleo-topography for the region of interest, according to an embodiment of the present disclosure. In the illustrated embodiment, a geological model 402 is provided as an initialization of the reconstruction process. Geological model 402 can include identified surface 208 below a plurality of geological layers, or overburden stratigraphy 404. Overburden stratigraphy 404 can represent the additional sedimentary units overlaid on the surface of interest in the modern era. A restoration process 406 can be performed on geological model 402, such that restoration, backstripping, and/or decompaction methods can restore identified surface 208 to a timespan during which identified surface 208 was exposed to the open air. Restoration process 406 can accordingly account for cross-correlated paleogeological eras in which weathering intensity was at a peak and during which identified surface 208 was exposed to weathering effects. As such, paleo-topographic map 408 can be constructed from restoration process 406 (e.g., via paleo-topographic reconstruction module 112) to provide paleo-highs and paleo-lows for identified surface 208.

FIG. 5 is an example superimposition of the various paleo-data and maps to construct a final paleo-topography with estimated historical flow, according to an embodiment of the present disclosure. In the illustrated embodiment, an initial drainage network 502 is depicted within a reconstructed paleo-topography. Initial drainage network 502 includes a plurality of stream lines 504 throughout, such that various flow patterns are discernable, divided by each catchment boundary 506. Stream lines 504 can represent areas of increased water flow and pooling, such that surface water was present at each of stream lines 504 during the desired timespan.

FIG. 5 further depicts a superimposed drainage network 508 formed (e.g., via paleo-data superimposition module 116) to include source rock 510 overlaid on the reconstructed paleo-topography and paleo-flow drainage network. One or more stream lines 504 can be identified that are within the same catchment boundaries 506 as source rock 510, and that intersect the location of source rock 510. Using the paleo-topography, one or more source-connected streams 512 can be identified and highlighted, through which water-dissolved minerals can flow away from source rock 510. Further, source-connected stream 512 and the paleo-topography can be utilized (e.g., via paleo-low and sink determination module 118) in the determination of an identified sink 514. Identified sink 514 can be a paleo-low in fluid communication with source-connected streams 512, such that the flow of source-connected stream 512 will pool and coalesce at identified sink 514. Accordingly, any water-dissolved minerals from source rock 510 can flow through source-connected stream 512 and aggregate within identified sink 514. Over time, identified sink 514 can thus become a repository of valuable minerals far from source rock 510 in which they formed.

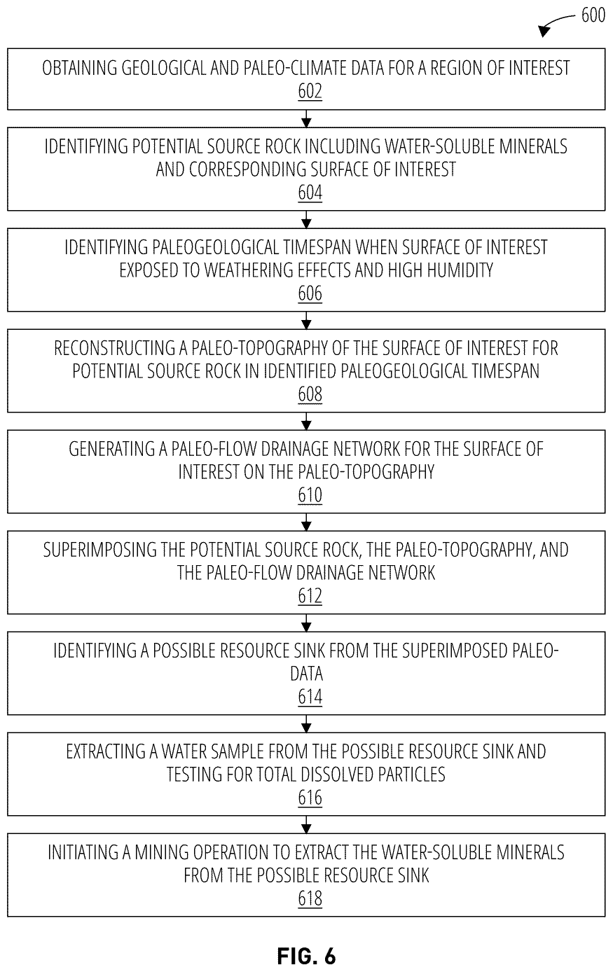

In view of the structural and functional features described above, example methods will be better appreciated with reference to FIG. 6. While, for purposes of simplicity of explanation, the example methods of FIG. 6 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

FIG. 6 illustrates a method 600 for identifying, testing, and mining resource sinks of water-soluble minerals developed from the weathering of potential source rocks with water-soluble minerals, according to one or more embodiments of the present disclosure (steps 602-618). Method 600 can be implemented by system 100, as shown in FIG. 1, and using the various paleo-data shown in FIGS. 2-5. As such, reference may be made to the examples of FIGS. 1-5 in the description of method 600. Method 600 can begin at 602 with obtaining geological and paleo-climate data for a region of interest. At 602, the obtained geological data can include both surface data and subsurface data, which can include surface geology regional maps, outcrop maps, imagery maps, seismic attributes, well logs, extracted cores, drill cuttings, or a combination thereof (e.g., input data 104). Further, the obtained paleo-climate data (e.g., paleo-climate input data 108) can include parameters such as historical humidity values or estimated precipitation levels, such that weathering intensity at various timespans can be identified.

Method 600 can continue at 604 with identifying one or more potential source rocks (e.g., via source rock identification module 106), the potential source rocks including water-soluble minerals. The one or more potential source rocks identified at 604 can further include a corresponding surface of interest on which each potential source rock can be provided. The corresponding surface of interest can represent a sedimentary unit with which the potential source rocks can have been exposed to weathering effects over time. Using the identified potential source rocks, method 600 can continue at 606 with identifying one or more paleogeological eras during which the surface of interest was exposed to weathering effects and high humidity (e.g., via weathering intensity module 110). At 606, the paleo-climate data can be utilized to determine these paleogeological eras of increased weathering, such that specific timespans are identified for higher leaching of minerals from the potential source rocks. Further, at 606, these paleogeological eras can be cross-correlated to geological data to determine the paleogeological eras in which the surface of interest was exposed to weathering effects and in which the paleo-climate data indicates increased weathering.

Method 600 can continue at 608 with reconstructing a paleo-topography of the surface of interest for the potential source rock in the identified paleogeological timespan (e.g., via paleo-topographic reconstruction module 112). At 608, the specified paleogeological timespan and input data can be utilized in restoration, backstripping, and/or decompaction methods to reconstruct a topographical map of the surface of interest during the paleogeological era. The reconstructed topographical map can thus represent the peaks and valleys of the Earth's surface during times of increased weathering when the potential source rocks are exposed to open air. Using the reconstructed paleo-topography, method 600 can continue at 610 with generating a paleo-flow drainage network for the surface of interest on the paleo-topography. The paleo-flow drainage network can include catchment boundaries, and streams therein, for the reconstructed paleo-topography. The paleo-flow drainage network generated at 610 (e.g., via paleo-flow drainage network module 114) can provide likely flow patterns and common bodies of water overlaid on the reconstructed paleo-topography.

Method 600 can continue at 612 with superimposing the potential source rock, the paleo-topography, and the paleo-flow drainage network onto a single map (e.g., via paleo-data superimposition module 116). The single map can enable the identification of mineral-rich streams at or near the potential source rock, as well as paleo-lows connected to the potential source rock via said mineral-rich streams. This single map, generated at 612 via superimposition, can be utilized at 614 for identifying a possible resource sink (e.g., via paleo-low and sink determination module 118). The possible resource sink can be identified at 612 such that the possible resource sink is a paleo-low area into which one or more mineral-rich streams flow in the paleo-flow drainage network. Following both the paleo-flow drainage network and the reconstructed paleo-topography, a sweetspot of possible minerals can be identified where any water-dissolved minerals can have coalesced and formed into rich deposits.

To verify the presence of valuable minerals, method 600 can continue at 616 with extracting a water sample from the possible resource sink and testing for total dissolved particles (e.g., via identified sink screener 120). The water sample extracted from the possible resource sink at 616 can be tested to assess a salinity thereof. At 616, the salinity can be extrapolated to determine an estimated value of total dissolved solids in the possible resource sink. The higher the value of total dissolved solids, the more likely the possible resource sink includes valuable minerals therein. Thus, method 600 can continue at 618 with initiating a mining operation to extract the water-soluble minerals from the possible resource sink. In some embodiments, initiation of the mining operation of the possible resource sink can be in response to the estimated value of total dissolved solids exceeding a pre-determined threshold, such that the pre-determined threshold indicates the likely presence of valuable minerals. As such, the pre-determined threshold can correlate to an acceptable risk for performance of mining operations.

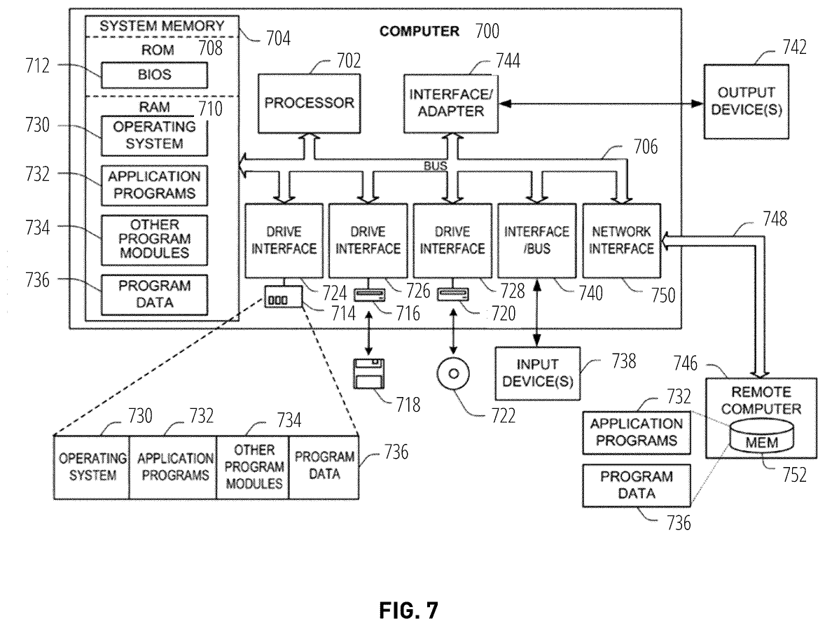

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 7. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device, or other IC (such as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium, or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 7 illustrates one example of a computer system 700, or electronic device, that can be employed to execute one or more embodiments of the present disclosure. Computer system 700 can be implemented on one or more general purpose networked computer systems, embedded computer systems, routers, switches, server devices, client devices, various intermediate devices/nodes or standalone computer systems. Additionally, computer system 700 can be implemented on various mobile clients such as, for example, a personal digital assistant (PDA), laptop computer, pager, and the like, provided it includes sufficient processing capabilities.

Computer system 700 includes processing unit 702, system memory 704, and system bus 706 that couples various system components, including the system memory 704, to processing unit 702. System memory 704 can include volatile (e.g., RAM, DRAM, SDRAM, Double Data Rate (DDR) RAM, etc.) and non-volatile (e.g., Flash, NAND, etc.) memory, such that system memory 704 can represent a non-transitory computer-readable medium. Dual microprocessors and other multi-processor architectures also can be used as processing unit 702. System bus 706 may be any of several types of bus structure including a memory bus or memory controller, a peripheral bus, and a local bus using any of a variety of bus architectures. System memory 704 includes read only memory (ROM) 708 and random access memory (RAM) 710. A basic input/output system (BIOS) 712 can reside in ROM 708 containing the basic routines that help to transfer information among elements within computer system 700.

Computer system 700 can include a hard disk drive 714, magnetic disk drive 716 (e.g., to read from or write to removable disk 718), and an optical disk drive 720 (e.g., for reading CD-ROM disk 722 or to read from or write to other optical media). Hard disk drive 714, magnetic disk drive 716, and optical disk drive 720 are connected to system bus 706 by a hard disk drive interface 724, a magnetic disk drive interface 726, and an optical drive interface 728, respectively. The drives and associated computer-readable media provide nonvolatile storage of data, data structures, and computer-executable instructions for computer system 700. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and ROM 708, including operating system 730, one or more application programs 732, other program modules 734, and program data 736. In some examples, the application programs 732 can include mineral sink identification engine 102, source rock identification module 106, weathering intensity module 110, paleo-topographic reconstruction module 112, paleo-low and sink determination module 118, paleo-flow drainage network module 114, paleo-data superimposition module 116, and identified sink screener 120. The program data 736 can include any of input data 104, paleo-climate input data 108, geological cross-section 200, weathering plot 300, geological model 402, paleo-topographic map 408, initial drainage network 502, superimposed drainage network 508, and any combination thereof. The application programs 732 and program data 736 can include functions and methods programmed to identify, test, and mine possible resource sinks from the weathering of potential source rocks including water-soluble minerals, such as shown and described herein.

A user may enter commands and information into computer system 700 through one or more input device 738, such as a pointing device (e.g., a mouse, touch screen), keyboard, microphone, joystick, game pad, scanner, and the like. These and other input devices 738 are often connected to processing unit 702 through a corresponding port interface 740 that is coupled to the system bus, but may be connected by other interfaces, such as a parallel port, serial port, or universal serial bus (USB). One or more output devices 742 (e.g., display, a monitor, printer, projector, or other type of displaying device) is also connected to system bus 706 via interface 744, such as a video adapter.

Computer system 700 may operate in a networked environment using logical connections to one or more remote computers, such as remote computer 746. Remote computer 746 may be a workstation, computer system, router, peer device, or other common network node, and typically includes many or all the elements described relative to computer system 700. The logical connections, schematically indicated at 748, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 700 can be connected to the local network through a network interface or adapter 750. When used in a WAN networking environment, computer system 700 can include a modem, or can be connected to a communications server on the LAN. The modem, which may be internal or external, can be connected to system bus 706 via an appropriate port interface. In a networked environment, application programs 732 or program data 736 depicted relative to computer system 700, or portions thereof, may be stored in a remote memory storage device 752.

In further embodiments, processing may be distributed among one or more processors at the same or different locations over a network. Processing steps 602-614 in method 600 and mineral sink identification engine 102 and its components (source rock identification module 106, weathering intensity module 110, paleo-topographic reconstruction module 112, paleo-low and sink determination module 118, paleo-flow drainage network module 114, paleo-data superimposition module 116, and identified sink screener 120) may be implemented on any type of computing device including, but not limited to, a laptop, desktop, tablet, workstation, mobile device or smartphone, kiosk, embedded system, or other computing device having at least one processor and a non-transitory computable readable memory. The computing device may include a browser, application, and operating system along with a user-interface depending upon a desired configuration. The computing device may have functionality performed at the same or different physical locations and by one or more processors located at the same or different locations. A computing device may also be coupled to one or more application programming interfaces (APIs) to perform or distribute aspects of the functionality described herein. Computing functionality as described herein may also be implemented on a server, cluster of servers, web server, cloud-computing platform, and/or other remote service. A client/server architecture may also be implemented as would be apparent to a person skilled in the art given this description.

Embodiments Disclosed Herein Include:
A. A computer-implemented method for identifying resource sinks of water-soluble minerals includes obtaining geological data and paleo-climate data for a region of interest, the geological data including surface data and subsurface data, identifying a potential source rock and a corresponding surface of interest from the geological data, the potential source rocks including water-soluble minerals, reconstructing a paleo-topography of the surface of interest for a paleogeological timespan in which the potential source rock is exposed to weathering via the paleo-climate data, generating a paleo-flow drainage network for the surface of interest to identify streams and catchment boundaries for the region of interest during the paleogeological era, and identifying a possible resource sink from the potential source rock, paleo-topography, and paleo-flow drainage network, wherein the possible resource sink is a paleo-low area into which one or more mineral-rich streams flows in the paleo-flow drainage network.
B. A system for locating and sampling resource sinks includes a mineral sink identification engine, implemented on at least one processor, operable to receive geological data and paleo-climate data to identify possible resource sinks. The mineral sink identification engine includes a source rock identification module operable to receive the geological data and identify potential source rocks for valuable minerals and corresponding surfaces of interest, a paleo-topographic reconstruction module operable to reconstruct a paleo-topography of the surfaces of interest wherein the potential source rocks are exposed to open air, a paleo-flow drainage network module operable to construct a paleo-flow drainage network of the surfaces of interest using the geological data and the paleo-climate data, and a paleo-low and sink determination module operable to utilize the potential source rocks, the paleo-topography, and the paleo-flow drainage network to identify the possible resource sinks. The system further includes an identified sink screener operable to extract and test a sample from each possible resource sink for an evaluation of valuable minerals therein.
C. A non-transitory computer-readable medium stores machine-readable instructions, which, when executed by a processor of an electronic device, cause the electronic device to identify a potential source rock and a corresponding surface of interest from available geological data, the potential source rocks including water-soluble minerals, reconstruct a paleo-topography of the surface of interest for a paleogeological timespan in which the potential source rock is exposed to weathering, and generate a paleo-flow drainage network for the surface of interest to identify streams and catchment boundaries for a region of interest during the paleogeological era. The machine-readable instructions further cause the electronic device to identify a possible resource sink from the potential source rock, paleo-topography, and paleo-flow drainage network, wherein the possible resource sink is a paleo-low area into which one or more mineral-rich streams flows in the paleo-flow drainage network, extract and assess, via an identified sink screener, a water sample from the possible resource sink to determine an estimated value of total dissolved solids in the possible resource sink, and initiate a mining operation of the possible resource sink in response to the estimated value of total dissolved solids exceeding a pre-determined threshold.

Each of embodiments A through C may have one or more of the following additional elements in any combination: Element 1: further comprising: superimposing the potential source rock, the paleo-topography of the surface of interest, and the paleo-flow drainage network onto a single map to identify mineral-rich streams at or near the potential source rock and paleo-lows connected to the potential source rock via said mineral-rich streams. Element 2: further comprising: extracting, via an identified sink screener, a water sample from the possible resource sink, and assessing a salinity of the water sample to determine an estimated value of total dissolved solids in the possible resource sink. Element 3: further comprising: initiating a mining operation of the possible resource sink in response to the estimated value of total dissolved solids exceeding a pre-determined threshold. Element 4: further comprising: identifying, via the paleo-climate data, one or more paleogeological eras of increased weathering for leaching of valuable minerals from the potential source rock, wherein the paleo-topography is reconstructed for the surface of interest to represent the one or more paleogeological eras. Element 5: wherein the paleogeological eras of increased weathering are identified via historical humidity values and estimated surface exposures of the potential source rock. Element 6: wherein the surface data includes surface geology regional maps, outcrop maps, imagery maps, or a combination thereof, and wherein the subsurface data includes seismic attributes, well logs, extracted cores, drill cuttings, or a combination thereof. Element 7: wherein the potential source rocks are volcanic rocks formed via igneous intrusions. Element 8: the mineral sink identification engine further including: a weathering intensity module operable to receive the paleo-climate data and identify paleogeological eras of increased weathering for leaching of valuable minerals from the potential source rocks.

Element 9: wherein the paleogeological eras of increased weathering are provided to the paleo-topographic reconstruction module to provide a paleogeological timespan to be reconstructed. Element 10: the mineral sink identification engine further including: a paleo-data superimposition module operable to receive paleo-data including the potential source rocks, the paleo-topography, and the paleo-flow drainage network, and operable to superimpose the paleo-data for use in the paleo-low and sink determination module. Element 11: wherein the identified sink screener is operable to extract a water sample from each possible resource sink and determine a salinity of each water sample to determine total dissolved solids in each water sample. Element 12: wherein the geological data further includes paleogeographic data, gross-depositional environment data, facies maps, or a combination thereof. Element 13: wherein the paleo-flow drainage network module is operable to receive the paleo-topography reconstructed via the paleo-topographic reconstruction module and construct the paleo-flow drainage network on the surfaces of interest in the paleo-topography. Element 14: wherein the instructions, when executed by the processor of the electronic device, further cause the electronic device to: superimpose the potential source rock, the paleo-topography of the surface of interest, and the paleo-flow drainage network onto a single map to identify mineral-rich streams at or near the potential source rock and paleo-lows connected to the potential source rock via said mineral-rich streams. Element 15: wherein the instructions, when executed by the processor of the electronic device, further cause the electronic device to: identify, via available paleo-climate data, one or more paleogeological eras of increased weathering for leaching of valuable minerals from the potential source rock, wherein the paleo-topography is reconstructed for the surface of interest to represent the one or more paleogeological eras. Element 16: wherein the available geological data comprises surface geology regional maps, outcrop maps, imagery maps, seismic attributes, well logs, extracted cores, drill cuttings, or a combination thereof. Element 17: wherein the instructions, when executed by the processor of the electronic device, further cause the electronic device to: test a salinity of the water sample, and extrapolate the estimated value of total dissolved solids in the possible resource sink from the salinity.

By way of non-limiting example, exemplary combinations applicable to A through C include: Element 1 with Element 2; Element 2 with Element 3; Element 3 with Element 4; Element 2 with Element 5; Element 1 with Element 6; Element 7 with Element 8; Element 9 with Element 10; Element 10 with Element 11; Element 10 with Element 12; and Element 10 with Element 13 . . . .

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A computer-implemented method for identifying resource sinks of water-soluble minerals, the method comprising:
obtaining geological data and paleo-climate data for a region of interest, the geological data including surface data and subsurface data;
identifying a potential source rock and a corresponding surface of interest from the geological data, the potential source rocks including water-soluble minerals;
reconstructing a paleo-topography of the surface of interest for a paleogeological timespan in which the potential source rock is exposed to weathering via the paleo-climate data;
generating a paleo-flow drainage network for the surface of interest to identify streams and catchment boundaries for the region of interest during the paleogeological era; and
identifying a possible resource sink from the potential source rock, paleo-topography, and paleo-flow drainage network, wherein the possible resource sink is a paleo-low area into which one or more mineral-rich streams flow in the paleo-flow drainage network.

2. The computer-implemented method of claim 1, further comprising:
superimposing the potential source rock, the paleo-topography of the surface of interest, and the paleo-flow drainage network onto a single map to identify mineral-rich streams at or near the potential source rock and paleo-lows connected to the potential source rock via said mineral-rich streams.

3. The computer-implemented method of claim 1, further comprising:
extracting, via an identified sink screener, a water sample from the possible resource sink; and
assessing a salinity of the water sample to determine an estimated value of total dissolved solids in the possible resource sink.

4. The computer-implemented method of claim 3, further comprising:
initiating a mining operation of the possible resource sink in response to the estimated value of total dissolved solids exceeding a pre-determined threshold.

5. The computer-implemented method of claim 1, further comprising:
identifying, via the paleo-climate data, one or more paleogeological eras of increased weathering for leaching of valuable minerals from the potential source rock,
wherein the paleo-topography is reconstructed for the surface of interest to represent the one or more paleogeological eras.

6. The computer-implemented method of claim 5, wherein the paleogeological eras of increased weathering are identified via historical humidity values and estimated surface exposures of the potential source rock.

7. The computer-implemented method of claim 1, wherein the surface data includes surface geology regional maps, outcrop maps, imagery maps, or a combination thereof, and wherein the subsurface data includes seismic attributes, well logs, extracted cores, drill cuttings, or a combination thereof.

8. The computer-implemented method of claim 1, wherein the potential source rocks are volcanic rocks formed via igneous intrusions.

9. A system for locating and sampling resource sinks, the system comprising:
a mineral sink identification engine, implemented on at least one processor, operable to receive geological data and paleo-climate data to identify possible resource sinks, the mineral sink identification engine including:
a source rock identification module operable to receive the geological data and identify potential source rocks for valuable minerals and corresponding surfaces of interest,
a paleo-topographic reconstruction module operable to reconstruct a paleo-topography of the surfaces of interest wherein the potential source rocks are exposed to open air,
a paleo-flow drainage network module operable to construct a paleo-flow drainage network of the surfaces of interest using the geological data and the paleo-climate data, and
a paleo-low and sink determination module operable to utilize the potential source rocks, the paleo-topography, and the paleo-flow drainage network to identify the possible resource sinks; and
an identified sink screener operable to extract and test a sample from each possible resource sink for an evaluation of valuable minerals therein.

10. The system of claim 9, the mineral sink identification engine further including:
a weathering intensity module operable to receive the paleo-climate data and identify paleogeological eras of increased weathering for leaching of valuable minerals from the potential source rocks.

11. The system of claim 10, wherein the paleogeological eras of increased weathering are provided to the paleo-topographic reconstruction module to provide a paleogeological timespan to be reconstructed.

12. The system of claim 10, the mineral sink identification engine further including:
a paleo-data superimposition module operable to receive paleo-data including the potential source rocks, the paleo-topography, and the paleo-flow drainage network, and operable to superimpose the paleo-data for use in the paleo-low and sink determination module.

13. The system of claim 9, wherein the identified sink screener is operable to extract a water sample from each possible resource sink and determine a salinity of each water sample to determine total dissolved solids in each water sample.

14. The system of claim 9, wherein the geological data further includes paleogeographic data, gross-depositional environment data, facies maps, or a combination thereof.

15. The system of claim 9, wherein the paleo-flow drainage network module is operable to receive the paleo-topography reconstructed via the paleo-topographic reconstruction module and construct the paleo-flow drainage network on the surfaces of interest in the paleo-topography.

16. A non-transitory computer-readable medium storing machine-readable instructions, which, when executed by a processor of an electronic device, cause the electronic device to:
identify a potential source rock and a corresponding surface of interest from available geological data, the potential source rocks including water-soluble minerals;
reconstruct a paleo-topography of the surface of interest for a paleogeological timespan in which the potential source rock is exposed to weathering;
generate a paleo-flow drainage network for the surface of interest to identify streams and catchment boundaries for a region of interest during the paleogeological era;
identify a possible resource sink from the potential source rock, paleo-topography, and paleo-flow drainage network, wherein the possible resource sink is a paleo-low area into which one or more mineral-rich streams flow in the paleo-flow drainage network;
extract and assess, via an identified sink screener, a water sample from the possible resource sink to determine an estimated value of total dissolved solids in the possible resource sink; and
initiate a mining operation of the possible resource sink in response to the estimated value of total dissolved solids exceeding a pre-determined threshold.

17. The non-transitory computer-readable medium of claim 16, wherein the instructions, when executed by the processor of the electronic device, further cause the electronic device to:
superimpose the potential source rock, the paleo-topography of the surface of interest, and the paleo-flow drainage network onto a single map to identify mineral-rich streams at or near the potential source rock and paleo-lows connected to the potential source rock via said mineral-rich streams.

18. The non-transitory computer-readable medium of claim 16, wherein the instructions, when executed by the processor of the electronic device, further cause the electronic device to:
identify, via available paleo-climate data, one or more paleogeological eras of increased weathering for leaching of valuable minerals from the potential source rock,
wherein the paleo-topography is reconstructed for the surface of interest to represent the one or more paleogeological eras.

19. The non-transitory computer-readable medium of claim 16, wherein the available geological data comprises surface geology regional maps, outcrop maps, imagery maps, seismic attributes, well logs, extracted cores, drill cuttings, or a combination thereof.

20. The non-transitory computer-readable medium of claim 16, wherein the instructions, when executed by the processor of the electronic device, further cause the electronic device to:
test a salinity of the water sample; and
extrapolate the estimated value of total dissolved solids in the possible resource sink from the salinity.
